(19) United States
(12) Patent Application Publication
(10) Pub. No.: US 2005/0228458 A1
(43) Pub. Date: Oct. 13, 2005

(54) Finishing wire assembly having variable insertion length and method therefor

(76) Inventors: Mo Jafari, Murrieta, CA (US); John Greenland, San Diego, CA (US); Frank E. Manning, Tamecula, CA (US); Charles Robert Peterson, Murrieta, CA (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/819,710

(22) Filed: Apr. 7, 2004

(51) Int. Cl. A61N 1/372

(52) U.S. Cl. 607/37

(57) ABSTRACT

A finishing wire assembly is provided herein that employs features that allow for the finishing wire assembly to be utilized with several different types of leads. Foe example, leads having a variety of different lengths and/or diameters, or other features that vary from lead to lead. The finishing wire assembly is adjustable in its length. For example, a portion of the finishing wire assembly is movable at a proximal portion of the finishing wire assembly, as further described below.

### TECHNICAL FIELD

Lead assemblies for conducting electrical signals to and from a location such as a heart, and more particularly, a wire and lead assembly allowing for placement, manipulation, or removal of placement devices.

### TECHNICAL BACKGROUND

Pacemaker leads are one example of a lead that represents the electrical link between the pulse generator and the heart tissue, which is to be excited and/or sensed. These pacemaker leads include single or multiconductors that are connected to an electrode in an electrode assembly at an intermediate portion or distal end of a pacing lead. A connector is included at the proximal end to form the electrical connection with the pacemaker.

To implant the lead within the patient, the lead is often fed intravenously toward the heart. The lead may be implanted within or travel through complex or tortuous vasculature using, for example, a guide catheter. After the lead has been placed in the desired position, the guide catheter must be extracted. However, extracting the guide catheter can cause the lead to be dislodged from the implanted position. A finishing wire can be used to assist in maintaining a lead in its implanted position during removal of the guide catheter. However, with leads having varying lengths, and varying terminal pins, requiring an individual finishing wire that is properly sized and supplied for each different lead type, size and length.

Accordingly, there is a need for a finishing wire assembly that can accommodate different sized leads having different features, such as different terminal pins.

### SUMMARY

A finishing wire assembly is provided herein that employs features that allow for the finishing wire assembly to be utilized with several different types of leads. For example, leads having a variety of different lengths and/or diameters, or other features that vary from lead to lead. The finishing wire assembly is used to stabilize a lead implanted within a patient, for example, within a heart, while a guide catheter is removed over the lead. In one option, the finishing wire assembly is adjustable in its length, for example, a portion of the finishing wire assembly is movable at a proximal portion of the finishing wire assembly, as further described below.

One embodiment of a finishing wire assembly includes a finishing wire that extends from a finishing wire proximal end to a finishing wire distal end, where the distal end has an optional atraumatic tip. The assembly further includes a portion that is movable relative to the finishing wire, for example, by sliding to modify the overall insertion length of the finishing wire. The portion can be secured to the finishing wire in several different ways. The assembly further includes a coupling that allows for the moving portion to be coupled with the lead.

Several options for the finishing wire assembly are as follows. For instance, in one option, the finishing wire assembly includes indicia disposed along the finishing wire that allow for a user to set the insertion length of the assembly. Optionally, a portion of the fixation portion engages the indicia, and provides feedback to the user.

The fixation portion can be secured to the finishing wire in a number of different manners. For instance, in one option, the fixation portion includes a set screw and housing. In another option, the fixation portion includes a collet assembly. In yet another option, the fixation portion includes a pin vice assembly, or a slider and grip assembly.

A method is also provided herein. The method includes advancing a guide catheter and a lead, for example, within a patient. A finishing wire assembly is provided, for example, including the ones discussed above. The finishing wire assembly is manipulated to modify an insertion length of the finishing wire. For example, a movable portion of the assembly is moved, for instance by sliding, and is coupled to a proximal portion of the lead. The finishing wire is inserted into the lead, and the guide catheter is removed over the finishing wire while the finishing wire is disposed within the lead.

Several options for the method are as follows. For instance, in one option, moving the movable portion along the finishing wire includes moving the movable portion to a location corresponding to indicia disposed along the finishing wire. In a further option, the method includes engaging a portion of the indicia with the movable portion.

Furthermore, several options for securing the moving portion are as follows. For instance, in one option, securing includes setting a set screw against the finishing wire. In another option, securing includes moving a portion of the fixation portion and causing an interference fit between a portion of the fixation portion and the finishing wire.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

FIG. 1 illustrates a block diagram of a finishing wire assembly and guide catheter and lead implanted within a heart and is constructed in accordance with one embodiment.

FIG. 2 illustrates a side elevational view of a finishing wire assembly as constructed in accordance with one embodiment.

FIG. 3 illustrates a side elevational view of a finishing wire assembly as constructed in accordance with one embodiment.

FIG. 4 illustrates a side elevational view of a finishing wire assembly as constructed in accordance with one embodiment.

FIG. 5 illustrates a cross sectional perspective view of a finishing wire assembly as constructed in accordance with one embodiment.

FIG. 6 illustrates a perspective view of a finishing wire assembly constructed in accordance with one embodiment.

FIG. 7 illustrates a perspective view of an adjusting finishing device as constructed in accordance with one embodiment.

### DESCRIPTION OF THE EMBODIMENTS

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

A finishing wire assembly is provided herein that employs features that allow for the finishing wire assembly to be utilized with several different types of leads. For example, leads having a variety of different lengths and/or diameters, or other features that vary from lead to lead. The finishing wire assembly is used to stabilize a lead implanted within a patient, for example, within a heart, while a guide catheter is removed over the lead. The finishing wire assembly is adjustable in its length, for example, a portion of the finishing wire assembly is movable at a proximal portion of the finishing wire assembly, as further described below.

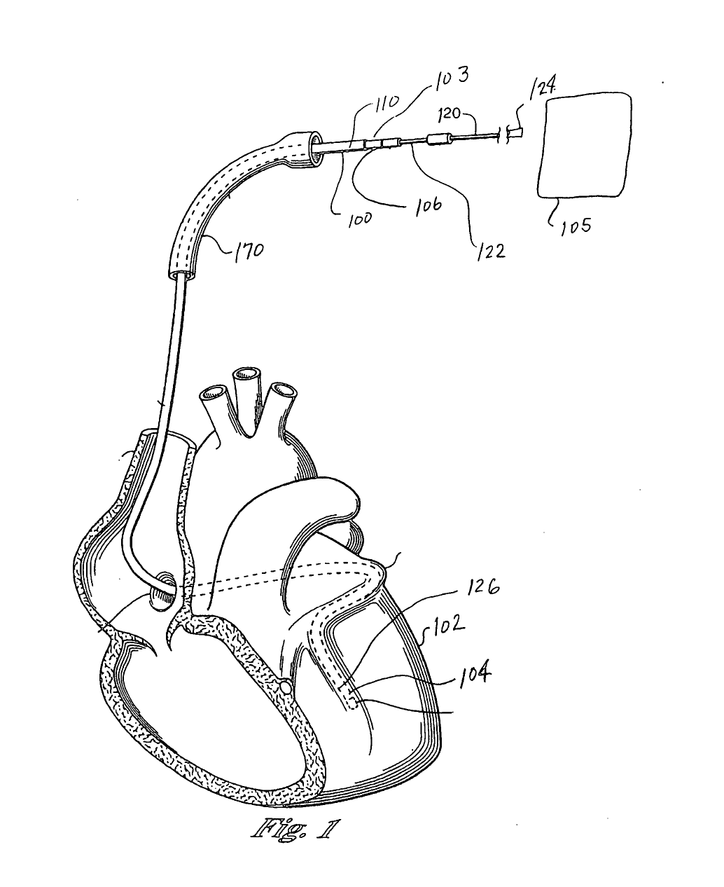

FIG. 1 illustrates a lead assembly constructed in accordance with one embodiment. A lead 100 includes a lead body 110 that is one option implanted in, on, or around a portion of a heart 102. The lead 100 extends from a proximal end 103 to a distal end 104 and provides a conduit of electrical signals in between the heart, and/or tissue to be excited and an energy source 105 such as a pulse generator. A terminal 106 provides the electrical connection and physical connection between the lead 100 and the energy source 105.

To implant the lead 100 within a heart 102, a guide catheter 170 is used. It should be noted other types of devices can be used as well, and are considered within the scope of this application. After the lead 100 is implanted with the guide catheter 170, the guide catheter 170 is to be removed from the patient. However, before removal, the guide wire is removed from the lead 100, and a finishing wire assembly 120 is disposed within the lead 100.

The finishing wire assembly 120 includes a finishing wire 122 that extends from a proximal end 124 to a distal end 126. When the finishing wire assembly 120 is disposed within the lead 100, the distal end 126 of the finishing wire 122 is disposed proximal to the distal end 104 of the lead 100. However, the finishing wire 122, in one option, is not disposed through the distal end 104 of the lead 100.

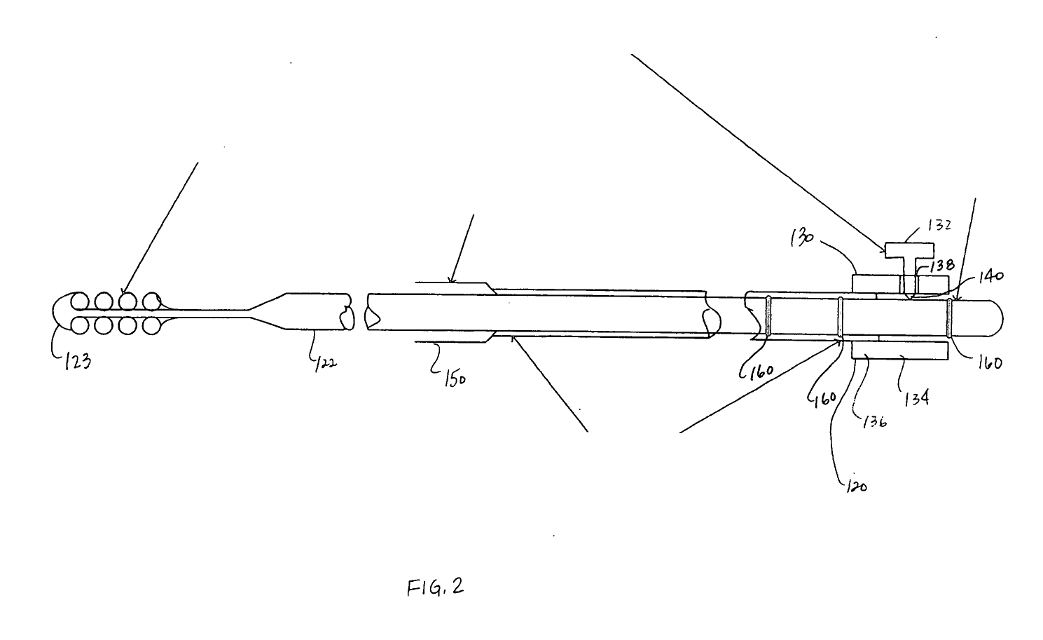

FIG. 2 illustrates an embodiment of the finishing wire assembly 120, in one example. The finishing wire assembly 120 further includes an adjustable assembly 130 that moves along the finishing wire 122, and is movably coupled with the finishing wire 122. The assembly 130 includes an actuator 132 that meets with a member 134, such as a housing 136. The assembly 130 is coupled with a coupling 150.

The coupling 150 is configured to meet with a portion of the terminal pin. In one option, the coupling 150 includes features that allow for the coupling 150 to be connected with terminal pins 106 (FIG. 1) having varying features and/or dimensions. One or more examples are listed in co-pending Patent Application entitled “FINISHING WIRE COMPATIBLE WITH MULTIPLE COMPONENTS”, attorney docket 279.731US1, filed on ______, which is incorporated herein by reference in its entirety.

The housing 136 and the coupling 150 are configured to move along the finishing wire 122, allowing for the portion of the length of the finishing wire 122 that is disposed within a lead, or the insertion length, to be adjusted in its length. This allows for the finishing wire assembly 120 to be used with multiple leads having multiple different lengths. In one option, the housing 136 slides along the finishing wire 122 and is fixated along various predetermined locations. In one option, the finishing wire 122 includes an indicator 160 thereon or other types of indicia to indicate the proper setting for each lead length.

The indicator 160 is, for example, a visual indicator whereby the user can align a portion of the housing 136 to abut a portion of the visual indicator. In another option, the indicator 160 provides a tactile indication and/or a visual indication, such that as the housing 136 is moved along the finishing wire 122, a portion of the finishing wire assembly 120 engages or interacts with the indicator 160. For example, the indicator 160 could include a projection that meets against a portion of, for example, the actuator 132. This provides feedback to the user when the actuator 132, and/or a portion of the housing 136 or the finishing wire assembly 120 has interacted with the indicator 160. Furthermore, the indicator 160 may include marks having different indicia to indicate the various leads and their respective lead lengths.

As mentioned above, the finishing wire assembly 120 allows for the overall length of the finishing wire 122 that is to be inserted into the lead, is adjustable in its insertion length. In one option, the finishing wire assembly includes a housing 136 that is movable along the finishing wire 122. The housing 136 can be secured into a place along the finishing wire 122 using an actuator 132. The actuator 132, and one option, includes a threaded component that is threadedly engaged with a portion of the housing 136. The housing includes an aperture 138 therein, with internally threaded portions therein. As the actuator 132 is threaded through the aperture 138, a portion 140 of the actuator 132 projects through the housing and engages a portion of the finishing wire 122. The projection 140 engages the finishing wire 122 and secures the housing 136 relative to the finishing wire 122. To further adjust the insertion length of the finishing wire 122 that is disposed within the lead, a user disengages the portion 140 from engagement with the finishing wire 122, allowing for the housing 136 to be moved along the finishing wire 122 and adjusting the overall length. Once the new position is selected by the user, the actuator 132 can be used to again secure the portion 140 into a new position along the finishing wire 122, by, for example, rotating the actuator 132.

As mentioned above, the overall insertion length of the finishing wire 122 is adjustable. The finishing wire 122 is disposed within the lead, however the distal portion of the finishing wire 122 is, in one option, not disposed through the end of the lead. In another option, the finishing wire 122 includes an atraumatic tip 123 at the distal portion of finishing wire 122. This alleviates the need to always insure that the finishing wire 122 is contained within the lead body for safety. The finishing wire 122 further can extend through the distal end of the lead without damaging tissue.

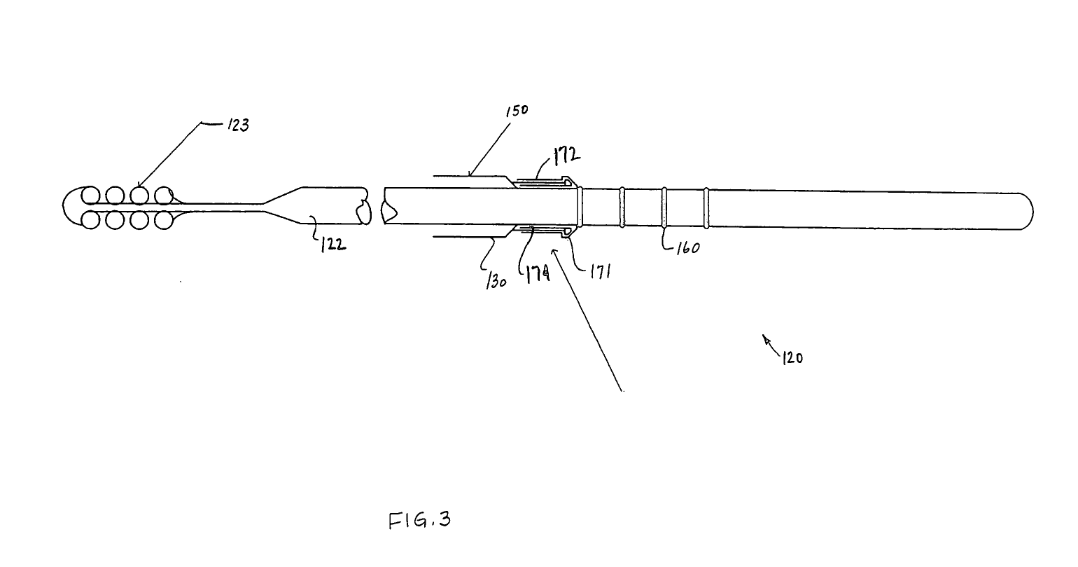

FIG. 3 illustrates another embodiment for the finishing wire assembly 120, that includes a finishing wire 122 and an assembly 130 that is movable relative to the finishing wire 122. Many of the elements and embodiments discussed above for the previous figure are incorporated for this figure as well, and will not be repeated herein. It should be noted that one or more of the features discussed and/or illustrated can be combined with this embodiment, or in further embodiments as discussed below. The finishing wire assembly illustrated in FIG. 3, similar to that shown in FIG. 2, is also adjustable in its overall insertion length, and can be used with multiple different leads without having to have multiple different finishing wire assemblies.

The finishing wire assembly 120, in one option, includes a collet assembly 171 that allows for the movable portion of the finishing wire assembly 120 to be secured into position along the finishing wire 122. As discussed above, the collet assembly is movable along the finishing wire 122, for example, the collet assembly 171 is slideable along the finishing wire 122, in order to change the overall insertion length of the finishing wire assembly 120. The collet assembly 171 includes an outer component 172 and an inner component 174, where the outer component 172 can be moved, for example, rotated, to secure the inner component 174 to the finishing wire 122. As the outer component 172 is manipulated, the inner component 174 clamps down or forms an interference fit with the finishing wire 122, along for the collet assembly 171 to be secured and placed along a position of the finishing wire 122.

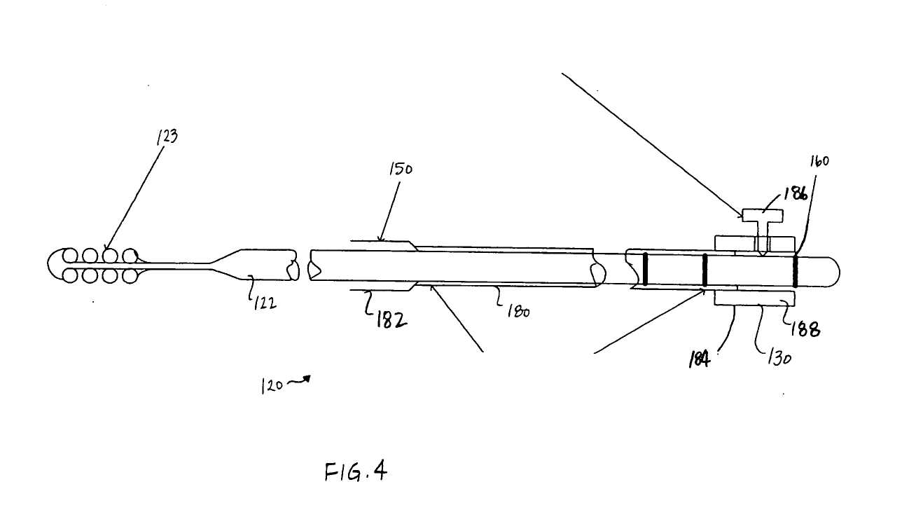

FIG. 4 illustrates another embodiment of the finishing wire assembly 120. The finishing wire assembly 120 again includes features that allow for the overall insertion length of the finishing wire 122 to be adjusted and customized for the type of lead it is used with. For example, the finishing wire assembly 120 includes a finishing wire 122 and an assembly 130 that is movable relative to the finishing wire 122. The finishing wire assembly 120 illustrated in FIG. 4 includes the features discussed above and below for the various embodiments and can be incorporated wholly or partially herein.

In one option, the finishing wire assembly 120 includes a hypo tube 180 that has a connector 182 disposed along one end. The connector 182 includes features that are able to fit terminal pins having varied dimensions, as discussed above. For example, in one option, the connector 182 includes features that allow for the connector 182 to connect with both LV-1 and IS-1 lead terminal pins. The connector 182 allows for the portion of the finishing wire assembly 120 to be coupled with a proximal portion of the lead, for example, with the terminal pin.

The hypo tube 180 extends from a proximal end to a distal end. The connector 182 is disposed at the distal end of the hypo tube 180. At the proximal end of the hypo tube 180 is a feature that allows for the hypo tube 180 to be secured along the finishing wire 122. In one option, an adjustable set screw assembly 184 is coupled with the proximal end of the hypo tube 180. The adjustable set screw assembly 184 includes a housing 188 and a set screw 186, allowing for the hypo tube 180 to be secured in a particular location along the finishing wire 122, and thereby allowing for adjustment of the insertion length of the finishing wire 122.

The set screw 186 is disposed through a portion of a housing 188 that allows for the set screw 186 to engage a portion of the finishing wire 122 and secure the adjustable set screw assembly 184 therewith. As discussed above, the finishing wire assembly 120 can include indicia 160 and/or markings to align the movable portion of the finishing wire assembly 120 in a particular location along the finishing wire 122.

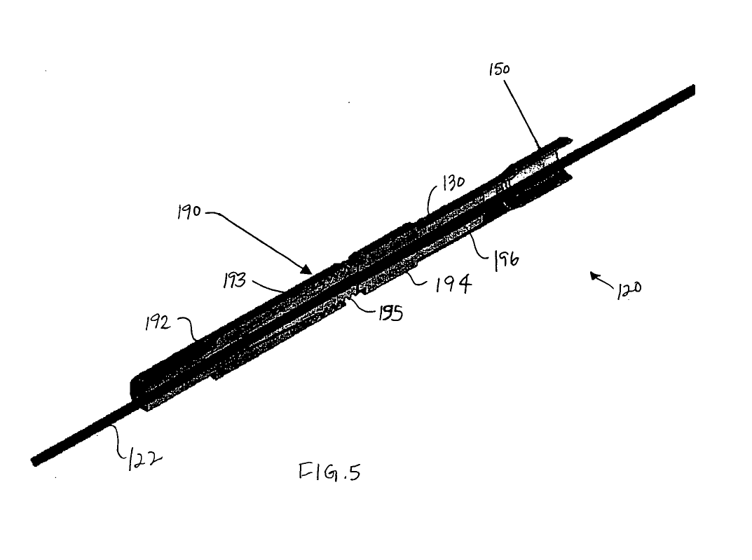

FIG. 5 illustrates another embodiment of the finishing wire assembly 120 that includes finishing wire 122 and an assembly 130 that moves and is adjustable relative to the finishing wire 122. The embodiments of the above-discussed figures and below-discussed figures are incorporated herein and the various features can be included either wholly or partially with this embodiment as well. The finishing wire assembly 122 further includes an adjustable pin vice assembly 190. The pin vice assembly 190 includes an actuator 192 that is used in conjunction with a clamping member 194. The clamping member 194 includes a surface such as an internal surface 196 that engages and abuts the outer diameter of the finishing wire 122. During use of the device the actuator 192 is used to clamp the clamping member 194 and the inner surface 196 of the clamping member 194 to be secured against the finishing wire 122. The inner surface 196 forms an interference fit with the finishing wire 122 and allows for the pin vice assembly 190 to be secured within a position along the finishing wire 122, and thereby adjusting the overall length of the finishing wire assembly 120.

In one option the actuator 192 includes internal threads 193 that interface with external threads 195 of the clamping member 194. The actuator 192 is tapered such that as the internal threads 193 engage the outer threads 195 of the clamping member 194, the inner surface 196 is caused to have a smaller and smaller inner diameter, thereby clamping onto the outer surface of the finishing wire 122. As the adjustable pin vice assembly 190 is moved along the finishing wire 122 and secured in position, this allows for the overall insertion length of the finishing wire 122 that is disposed within the lead to be manipulated and changed. The adjustable pin vice assembly 190 further includes a connector 150 that allows for the pin vice assembly and the finishing wire assembly 120 to be connected with the terminal pin, for example, multiple types of terminal pins such as IS-1 and LV-1 terminal pins as further described above.

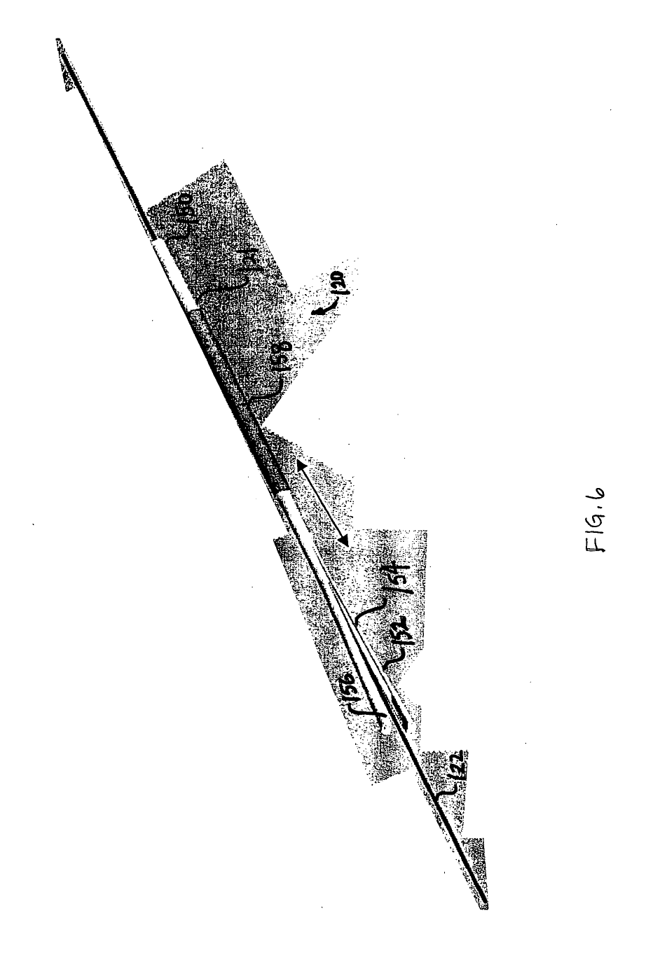

FIG. 6 illustrates another embodiment of a finishing wire assembly 120. The finishing wire assembly 120 includes a portion 121 that moves relative to the finishing wire 122, but also can secure the assembly to the finishing wire 122. The finishing wire assembly 120 also includes the finishing wire 122. The portion 121, such as a fixturing portion, can be moved along the finishing wire 122 and includes a connector portion 150 that allows for the finishing wire assembly 120 to be connected to terminal pins, such as IS-1 and LV-1, as further discussed above. The fixturing portion 121, similar to the assembly 130 (FIG. 5) discussed above, is allowed to move along the finishing wire 122 such that the overall length can be adjusted. Once the overall length is selected, as discussed above, the fixturing portion 121 is secured to the finishing wire 122. As further discussed above, the finishing wire assembly 120 can further include indicia to allow for the fixturing portion 121 to be disposed in a predetermined location that corresponds with a particular type of lead and its lead length. [00331 In one option, the fixturing portion 121 includes a gripping portion 152, and a slider 158. The gripping portion 152 allows for the fixturing portion 121 to be clamped to the finishing wire 122 at a particular location along the finishing wire 122. The slider 158 allows for actuation of the gripping mechanism by sliding there along.

The gripping portion 152 includes a first grip 154 and a second grip 156, for example, on opposite sides of the finishing wire 122. It should be noted that one or more gripping portions can be used in conjunction with this fixturing portion 121.

During use of the device, the slider 158 is slid along the longitudinal axis of the finishing wire 122 to slide until the grips 154 and 156 clamp down onto the finishing wire 122. As the slider 158 is slid towards the grips, the grips clamp down onto the finishing wire 122. In one option, the slider 158 is formed of a hypo tube. The slider 158 has a portion with an internal diameter that is sized such that as it is moved over the grips and the finishing wire 122 that the grips 154, 156, are compressed down onto the finishing wire 122. The finishing wire 122 is retained in an interference fit by the grips 154, 156. The slider 158 is retained at an interference fit with an outer portion of the grips 154, 156 to retain the grips in a clamped position. This allows for the fixturing portion 121 to be secured into a particular position along the finishing wire 122. This allows for the overall length to be determined by the user. To release the fixturing portion 121 from griping the finishing wire 122, the slider 158 is slid away from the grips 154, 156 to release the grips from engagement of the finishing wire 122.

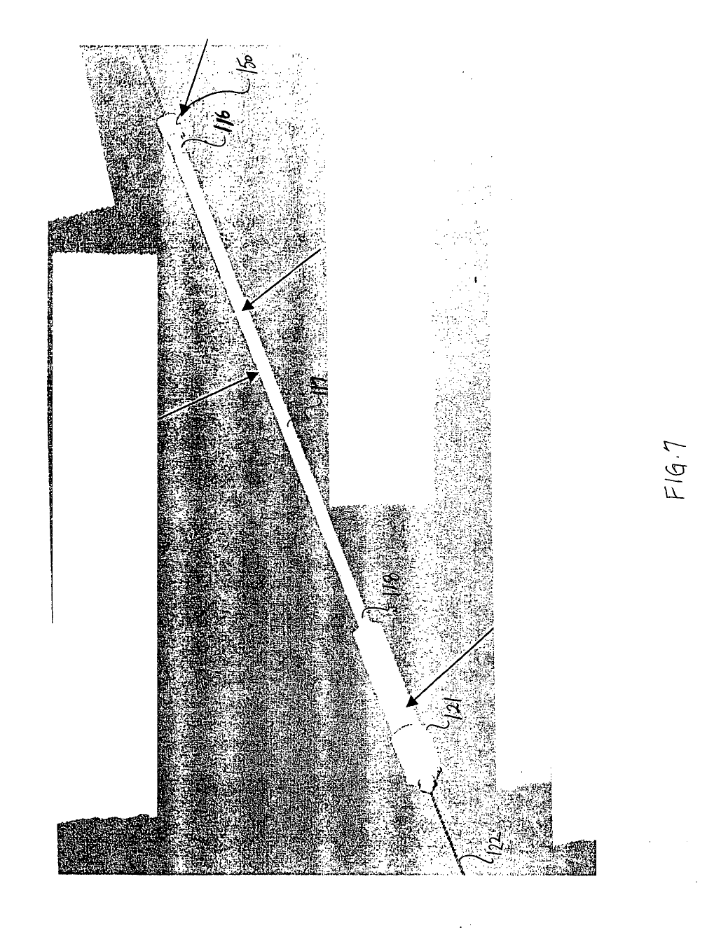

FIG. 7 illustrates another embodiment for an adjustable finishing device. The adjustable finishing device includes a fixed ring portion 121 that is movable relative to a finishing wire 122. The fixturing portion 121 can include any of the gripping devices discussed above in the earlier discussed embodiments. The adjustable finishing device includes a long tubular portion, that extends from a proximal end 118 to a distal end 116. Disposed of the proximal end 118 is the fixturing portion 121, and disposed at the distal end 116 is the connector 150 that can be used, for example, with multiple types of terminal pins. The long tubular portion is, in one option, sufficiently long in length to allow a guide catheter to be removed from the patient without disturbing the connection of the terminal pin 106 and the finishing wire 122. In one option, the length of the long tubular portion is long enough to allow guide catheters having a variety of lengths, for example, to be removed from the patient without disturbing the connection of the terminal pin 106 and the finishing wire 122.

The adjustable finishing device includes a sheath disposed between the proximal and distal ends 118, 116. The tubular portion can include, for example, a polymer sheath, or a hypo tube. The intermediate portion 117 is sized to receive the guiding catheter thereover, for example, as the guiding catheter is removed from the patient.

During use of the assemblies discussed above, a method includes advancing a guide catheter and a lead, for example, within a patient. The guide catheter, for example, is advanced through the vasculature of a patient, through use of a guide wire. The guide wire and the guide catheter further assists in advancing the lead through the vasculature. Once the lead has been positioned into place within the patient, the guide wire is removed from the lead.

A finishing wire assembly is provided, for example, including the ones discussed above. The finishing wire assists in maintaining the lead in its implanted position while the guide catheter is removed thereover. The finishing wire assembly is manipulated to modify an insertion length of the finishing wire. For example, a movable portion of the assembly is moved, for instance by sliding, and is coupled to a proximal portion of the lead. The movable portion of the assembly is moved along the finishing wire to the desired insertion length corresponding to the particular type of lead having a particular length. In one option, the assembly includes indicia that the user can use in determining the appropriate length to set the movable portion. In one option, the movable portion engages a portion of the indicia, for example, to provide tactile feedback to the user as to the indicia. Once the movable portion has been properly positioned, the movable portion is secured into position.

The movable or fixation portion can be secured to the finishing wire in a number of different manners. For instance, in one option, securing includes setting a set screw against the finishing wire. The user rotates or otherwise sets the set screw into position against the finishing wire to retain a housing member of the set screw assembly into position. In another option, securing includes moving a portion of the fixation portion and causing an interference fit between a portion of the fixation portion and the finishing wire. For example, an outer member is rotated, slid, or otherwise manipulated to collapse or reduce an inner portion of the device around the finishing wire. This reduces the inner dimensions of the inner portion around the finishing wire, causing the inner portion to grip the finishing wire, for example in an interference fit. The finishing wire is inserted into the lead, and the guide catheter is removed over the finishing wire while the finishing wire is disposed within the lead.

Advantageously, the adjustable finishing assembly can be used universally for multiple different means having multiple different lengths, while only requiring one type of device. This will aid in inventory control and manufacturing costs as multiple products will not need to be reduced and/or distributed for the varying length leads.

It is to be understood that the above description is intended to be illustrative, and not restrictive. Although the use of the implantable device has been described for use as a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

1. A finishing wire assembly comprising:
a finishing wire extending from a finishing wire proximal end to a finishing wire distal end and having a finishing wire intermediate portion therebetween;
a fixation portion movably coupled with the finishing wire, the fixation portion movable relative to the finishing wire, the fixation portion including a coupling couplable with a lead terminal pin; and
the fixation portion securable at two or more locations along the finishing wire intermediate portion.

2. The finishing wire assembly as recited in claim 1, further comprising indicia disposed along the finishing wire, the indicia having predetermined locations indicating various lengths for an insertion length.

3. The finishing wire assembly as recited in claim 2, wherein a portion of the fixation portion engages the indicia.

4. The finishing wire assembly as recited in claim 1, wherein the fixation portion includes a set screw and housing.

5. The finishing wire assembly as recited in claim 1, wherein the fixation portion includes a collet assembly.

6. The finishing wire assembly as recited in claim 1, wherein the fixation portion includes a pin vice assembly.

7. The finishing wire assembly as recited in claim 1, wherein the fixation portion includes a slider and grip assembly.

8. The finishing wire assembly as recited in claim 1, wherein the finishing wire distal end includes an atraumatic tip.

9. The finishing wire assembly as recited in claim 1, further comprising means for coupling the coupling with lead terminal pins having different inner and/or outer diameters.

10. The finishing wire assembly as recited in claim 1, wherein the fixation portion is slidable relative to the lead terminal pin.

11. A finishing wire assembly comprising:
a finishing wire extending from a finishing wire proximal end to a finishing wire distal end and having a finishing wire intermediate portion therebetween; and
means for movably coupling a fixation portion with the finishing wire, the fixation portion movable from a first position along the finishing wire to a second position along the finishing wire, the fixation portion securable along a portion of the finishing wire; and
the fixation portion including a coupling couplable with a lead terminal pin.

12. The finishing wire assembly as recited in claim 11, further comprising means for coupling the portion with terminal pins having different inner and/or outer diameters.

13. The finishing wire assembly as recited in claim 11, wherein the finishing wire distal end includes an atraumatic tip.

14. The finishing wire assembly as recited in claim 11, wherein the fixation portion is slidable relative to the finishing wire.

15. The finishing wire assembly as recited in claim 11, further comprising indicia disposed along the finishing wire, the indicia having predetermined locations indicating various insertion lengths.

16. A method comprising:
advancing a guide catheter;
advancing a lead over a guide wire through the guide catheter;
removing the guide wire from the lead;
modifying an insertion length of a finishing wire of a finishing wire assembly;
inserting at least a portion of the insertion length of the finishing wire into the lead after removing the guidewire; and
removing the guide catheter over the finishing wire while the finishing wire is disposed within the lead.

17. The method as recited in claim 16, further comprising moving a fixation portion along the finishing wire of the finishing wire assembly.

18. The method as recited in claim 16, wherein moving the fixation portion includes sliding the fixation portion along the finishing wire.

19. The method as recited in claim 16, further comprising coupling a portion of the fixation portion with a proximal portion of the lead.

20. The method as recited in claim 16, wherein modifying the insertion length of the finishing wire includes moving the fixation portion along the finishing wire, and securing the fixation portion at a location along the finishing wire.

21. The method as recited in claim 20, wherein securing the fixation portion includes setting a set screw against the finishing wire.

22. The method as recited in claim 20, wherein securing the fixation portion includes moving a portion of the fixation portion and causing an interference fit between a portion of the fixation portion and the finishing wire.

23. A method comprising:
advancing a guide catheter and lead;
providing a finishing wire assembly including a finishing wire and a movable portion;
moving the movable portion along the finishing wire to a location along the finishing wire;
securing the movable portion at the location along the finishing wire;
inserting at least a portion of the finishing wire into the lead and coupling the movable portion with the lead; and
removing the guide catheter over the finishing wire assembly while the finishing wire is disposed within the lead.

24. The method as recited in claim 23, wherein moving the movable portion along the finishing wire includes moving the movable portion to a location corresponding to indicia disposed along the finishing wire.

25. The method as recited in claim 24, further comprising engaging a portion of the indicia with the movable portion.

26. The method as recited in claim 23, wherein removing the guide catheter includes moving the guide catheter on to a portion of the movable portion.

27. The method as recited in claim 23, wherein moving the fixation portion includes sliding the fixation portion along the finishing wire.

28. The method as recited in claim 23, wherein securing the fixation portion includes setting a set screw against the finishing wire.

29. The method as recited in claim 23, wherein securing the fixation portion includes moving a portion of the movable portion and causing an interference fit between a portion of the fixation portion and the finishing wire.
